(19) United States
(12) Patent Application Publication
(10) Pub. No.: US 2017/0231539 A1
(43) Pub. Date: Aug. 17, 2017

(54) DEVICES AND METHODS FOR FLUID SAMPLE COLLECTION AND DIAGNOSTIC TESTING

(71) Applicant: HOOPE TECHNOLOGIES CORPORATION, Wilmington, DE (US)

(72) Inventors: Ernesto RODRIGUEZ LEAL, Monterrey (MX); Damel MEKTEPBAYEVA, Astana (KZ); Irina RYMSHINA, Lima (PE)

(73) Assignee: HOOPE TECHNOLOGIES CORPORATION, Wilimington, DE (US)

(21) Appl. No.: 15/504,278

(22) PCT Filed: Aug. 14, 2015
(86) PCT No.: PCT/US2015/045296

§ 371 Date: Feb. 15, 2017

Related U.S. Application Data
(60) Provisional application No. 62/037,619, filed on Aug. 15, 2014.

(51) Int. Cl. A61B 5/157; A61B 5/151; A61B 5/1468; A61B 5/15

(52) U.S. Cl. CPC A61B 5/157; A61B 5/150412; A61B 5/15192; A61B 5/150022; A61B 5/150267; A61B 5/150854; A61B 5/150862; A61B 5/15087; A61B 5/1468

(57) ABSTRACT

A medical device including a housing, and a diagnostic cartridge removably coupled to the housing, the diagnostic cartridge including a retractable needle mechanism disposed within the diagnostic cartridge, the retractable needle mechanism configured to extract a fluid sample, a fluid collection chamber disposed the retractable needle mechanism, the fluid collection chamber configured to collect the fluid sample extracted by the retractable needle mechanism, and a diagnostic chip disposed within the fluid collection chamber configured to analyze the fluid sample.

### CROSS-REFERENCE TO RELATED APPLICATIONS

This application is claims benefit of priority from Provisional U.S. Patent Application No. 62/037,619, filed Aug. 15, 2015, the contents of which are incorporated by reference in their entirety.

### FIELD

This application relates to medical devices, computing devices coupled to medical devices, and diagnostic methods using the medical devices, more specifically, medical devices for administering fluids to, or extracting fluids from, a user.

### BACKGROUND

World Health Organization statistics indicate that 500 million people worldwide become infected each year with one or more of the four most common sexually transmitted diseases (STDs): syphilis, gonorrhea, trichomoniasis, and chlamydia infection. These diseases generally have not outward symptoms for an extended period of time after initial infection. In fact, often these diseases are first diagnosed during family planning clinics, rather that presenting symptoms of infection. However, these diseases have been shown to cause potential infertility, cancer, and increased risk of contracting HIV, and in the case of pregnancy, high risk of stillbirth or miscarriage.

In many countries, clinic based STD testing has been largely unavailable, geographically inaccessible, expensive and embarrassing in developing countries. Further, at-home STD testing has been largely limited to populated urban areas in developing countries due to a lack of infrastructure. Often at-home testing options have not involved self-use kits, but instead have required a nurse or other medical professional to visit the patient's home to perform sample collection (i.e. collection of fluid samples such as blood, etc. for testing). As such, testing can be difficult to arrange at night or on weekends. As a result, working young adults, who represent the group most at-risk, may be discouraged from seeking out regular testing. Further, making results available by phone can create privacy concerns because ID cards cannot be physically checked, and patient authentication can be difficult. Further, the immunoassays often used for diagnosis can take 2-4 days for results to be available, potentially postponing needed treatment. Some known fluid (i.e. blood, lymph, etc.) collection tools and methods have involved using a separate lancet device that is inserted into the body to puncture the skin and release the fluid.

### SUMMARY

Aspects of example implementations of the present application may relate to a medical device for puncturing skin or other tissue to obtain a fluid sample and perform diagnostic tests on a collected sample. Aspects of example implementations of the present application may also relate to methods, apparatuses, and non-transitory computer readable media for medical diagnosis based on a collected sample.

The subject matter may include medical devices including a housing and a diagnostic cartridge removably coupled to the housing, the diagnostic cartridge having a retractable needle mechanism disposed within the diagnostic cartridge, the retractable needle mechanism configured to extract a fluid sample, a fluid collection chamber disposed the retractable needle mechanism, the fluid collection chamber configured to collect the fluid sample extracted by the retractable needle mechanism, and a diagnostic chip disposed within the fluid collection chamber configured to analyze the fluid sample.

The subject matter may also include medical devices including a housing, a first electrode disposed on the housing, the first electrode configured to contact a first portion of a user, a second electrode disposed on the housing a distance from the first electrode, the second electrode configured to contact a second portion of a user, a retractable needle mechanism disposed between the first electrode and the second electrode, the retractable needle mechanism configured to extract a fluid sample, and a power source configured to provide a voltage between the first electrode and the second electrode.

Additionally, the subject matter may include medical devices including a retractable needle mechanism configured to extract a fluid sample, the retractable needle mechanism having a needle, a needle housing surrounding the needle, an injector housing disposed adjacent to the needle housing, a biasing member disposed within the injector housing, the biasing member configured to apply a biasing force to the injector housing to direct the needle toward a target site, a retaining housing disposed adjacent the needle housing and opposite the injector housing, the retaining housing configured to engage the needle housing and retain the needle in a retracted position, a triggering mechanism provided on an exterior of the injector housing, wherein the injector housing is configured to move toward the needle housing in response to an external force being applied to the triggering mechanism, wherein the biasing member is configured to provide an increased biasing force to the needle housing in response to the injector housing moving toward the needle housing, wherein the needle housing is configured to move relative to the retaining housing in response to the increased biasing force exceeding a threshold, and wherein the needle is configured to pierce the target site in response to the needle housing moving relative to the retaining housing.

The subject matter may also include computing devices coupled to a medical device. The computing devices may include a processor and a memory storing computer instructions for controlling the computing device to perform receiving, from the medical device, data indicative of a detected at least one of a chemical, an antigen, an antibody, or a protein, identifying, based on the received data, diagnosis information indicative of a potential medical condition, and providing, based on the identified diagnosis information, follow-up information related to the potential medical condition.

Additionally, the subject matter may include methods for diagnosing a condition including receiving, from a medical device, data indicative of a detected at least one of a chemical, an antigen, an antibody, or a protein, identifying, by a computing device, diagnosis information indicative of a potential medical condition based on the received data, and providing, by a computing device, follow-up information related to the potential medical condition based on the identified diagnosis information.

The subject matter may also include non-transitory computer readable media having stored therein a program for making a computer execute a method for diagnosing a condition, said program including computer executable instructions for performing receiving data indicative of a detected at least one of a chemical, an antigen, an antibody, or a protein, identifying diagnosis information indicative of a potential medical condition based on the received data, and providing follow-up information related to the potential medical condition based on the identified diagnosis information.

### BRIEF DESCRIPTION OF THE DRAWINGS

A general architecture that implements features of the disclosure will now be described with reference to the drawings. The drawings and the associated descriptions are provided to illustrate implementations of the disclosure and not to limit the scope of the disclosure. Through the drawings, reference numbers are reused to indicate correspondence between referenced elements.

FIGS. 1A and 1B illustrate perspective views of a medical device according to an example implementation of the present application.

FIG. 2 illustrates a schematic view of an example implementation of the medical device illustrated in FIG. 1.

FIG. 3 illustrates an exploded view of another example implementation of the medical device illustrated in FIG. 1.

FIG. 4 illustrates an enlarged, internal view of a retractable needle mechanism of an example implementation of the medical device illustrated in FIG. 3.

FIGS. 5A-5F illustrate example implementations of a diagnostic chip used in an example implementation of the medical device illustrated in FIG. 1.

FIG. 6 illustrates an operational diagram of the medical device illustrated in FIGS. 1 and 2.

FIG. 7 shows an example environment suitable for some example implementations.

FIG. 8 shows an example computing environment with an example computing device suitable for use in some example implementations.

FIG. 9 illustrates a flowchart of a diagnostic method according to an example implementation of the present application.

### DETAILED DESCRIPTION

The subject matter described herein is taught by way of example implementations. Various details may have been omitted for the sake of clarity and to avoid obscuring the subject matter. The examples shown below are directed to apparatuses and structures for implementing fluid sample collection and diagnostic testing.

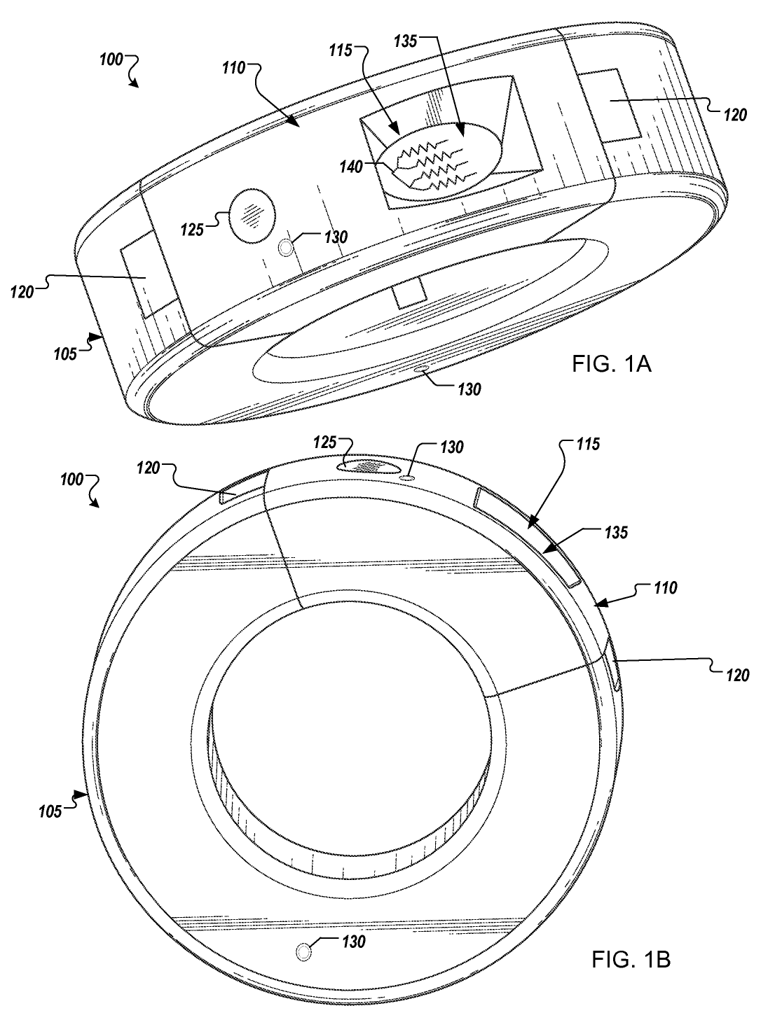

FIGS. 1A and 1B illustrate perspective views of a medical device 100 according to an example implementation of the present application. As illustrated, the medical device 100 may have a generally annular shape. The medical device 100 may include a wearable housing 105 and a cartridge 110 removable coupled to the wearable housing 105. The wearable housing 105 may have at least a partial ring structure such that the wearable housing 105 alone or in combination with the cartridge 110 may be configured to encircle (e.g. extend around) a portion of a human body. In some implementations, the wearable housing 105 may be sized to encircle a finger. However, example implementations of the present application are not limited to this configuration. For example, the wearable housing 105 may be sized to encircle a wrist, a forearm, an upper arm, a thigh, a shin, an ankle, or any other part of a human body.

The cartridge 110 may removably couple to the wearable housing 105 by one or more connectors 120. The connectors 120 may provide one or more of: (1) a physical connection to secure the cartridge 110 to the wearable housing 105, (2) an electrical connection to allow transmission of electrical current (such as direct current (DC) or alternating current (AC)) between the wearable housing 105 and the cartridge 110, and (3) a communication connection to allow transmission of signals between the wearable housing 105 and the cartridge 110. The connectors 120 are not particular limited but may include snap connectors, press-fit connectors, magnetic connectors or any other type of connector that may be apparent to a person of ordinary skill in the art.

The cartridge 110 may also include an opening 115 formed in an outer surface of the cartridge 110. The opening 115 may allow access or viewing of a fluid collection chamber 135 formed within the cartridge 110. As discussed in greater detail below, the fluid collection chamber 135 may have one or more diagnostic chips 140 that perform diagnostic analysis on fluid collected within the fluid collection chamber 135. In some example implementations, the opening 115 may be covered by a transparent window (not shown) or the opening 115 may be omitted (such that the fluid collection chamber 135 is fully enclosed) to prevent collected fluid leakage. The cartridge 110 may also include a triggering mechanism 125, such as a button, a switch, or other triggering mechanism that may be apparent to a person of ordinary skill in the art, that may be used to actuate a retractable needle mechanism as discussed below. In some embodiments, the cartridge 110 may be disposable and be thrown away after testing.

One or more indicators 130 may be located on the exterior of the medical device 100 to provide information to a user. The indicator 130 may provide status information (such as “ready for testing,” “testing complete,” etc.), diagnostic information (such as “test positive,” “test negative,” etc.) or any other information that may be apparent to a person of ordinary skill in the art. The indicator 130 may be a light source (such as a light bulb, an LED, etc.), an audio indicator (such as a speaker, etc.), or any other type of indicator that may be apparent to a person ordinary skill in the art. As illustrated, one indicator 130 may be provided on the wearable housing 105 and another indicator 130 may be provided on the cartridge 110. However, example implementations are not limited to this configuration. For example, one indicator 130 may be provided only on the wearable housing 105, one indicator 130 may be provided only on the cartridge 110, or more than two indicators 130 may be provided on the medical device 100.

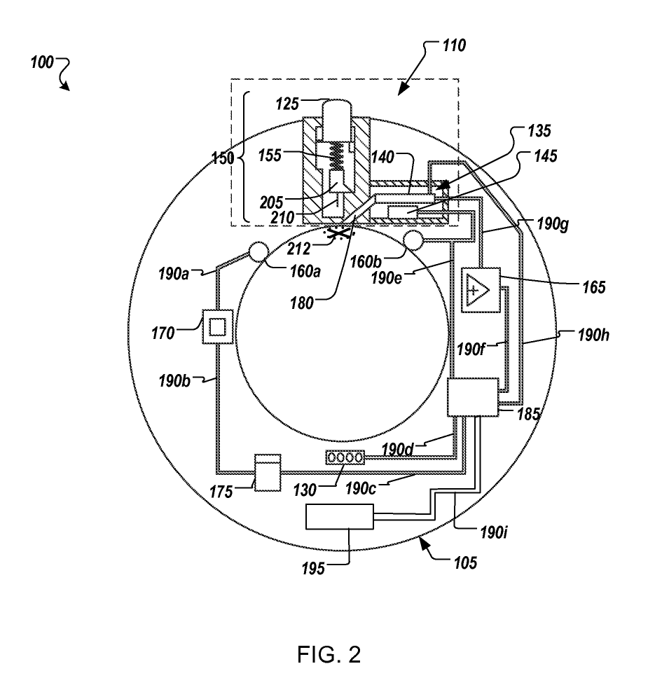

FIG. 2 illustrates a schematic view of the example implementation of the medical device 100 illustrated in FIG. 1. Again, as illustrated, the medical device 100 may have a generally annular shape and may include a wearable housing 105 and a cartridge 110 removable coupled to the wearable housing 105. In the schematic view of FIG. 2, internal components of the wearable housing 105 and the cartridge 110 are illustrated. The distribution of components between the wearable housing 105 and the cartridge 110 are not limited to the configuration illustrated and alternative configurations may be apparent to a person of ordinary skill in the art. For example, components illustrated as being part of the wearable housing 105 may instead be incorporated into the cartridge 110. Conversely, components illustrated as being part of the cartridge 110 may instead be incorporated into the wearable housing 105. Further, in some example implementations, all illustrated components of the cartridge 110 may be incorporated into the wearable housing 105 and the cartridge 110 may be omitted. Similarly, all components of the wearable housing 105 may be incorporated into the cartridge 110, and the wearable housing 105 may be omitted.

As illustrated in FIG. 2, the cartridge 110 may include the fluid collection chamber 135, a diagnostic chip 140, a power source 145, a retractable needle mechanism 150, and a capillary collection tube 180. The wearable housing 105 is illustrated as including an indicator 130, a pair of electrodes 160a and 160b, an amplifier 165, a transformer 170, a power transistor 175, a processor 185, and a plurality of electrical conductors 190a-190h interconnecting the components.

In the cartridge 110, the retractable needle mechanism 150 includes the triggering mechanism 125, a biasing member 155, a needle 210, and a needle housing 205. The triggering mechanism 125 is illustrated as a button located above the biasing member 155. The needle housing 205 and needle 210 are located below the biasing member 155. The retractable needle mechanism 150 is configured to transmit downward movement of the triggering mechanism 125 into downward movement of the needle 210. As discussed in greater detail below, the needle 210 is designed to pierce the skin of a user wearing the medical device 100 to release fluid (such as blood cells, lymph fluid, or any other fluid that may be apparent to a person of ordinary skill in the art). For reference, an area directly below the needle 210 (referred to herein as a target site 212) is illustrated to show a general area where the needle 210 may pierce the skin of the user wearing the medical device 100 to release the fluid.

The capillary collection tube 180 is configured to collect the released fluid from the target site 212 and transport the fluid into the fluid collection chamber 135 wherein it is analyzed using the diagnostic chip 140. As discussed in greater detail below, the diagnostic chip 140 may use lab-on-a-chip techniques to analyze the collected fluid to detect one or more chemicals, antigens, or proteins found in the sample. The power source 145 powers the diagnostic chip 140 via the electrical conductors 190a-190h. In some implementations, the power source 145 may be a battery, a fuel cell, or any other power source that may be apparent to a person of ordinary skill in the art. Further, in some example implementations, the power source may be a rechargeable power source or a one-time use power source.

In the wearable housing 105, each of the pair of electrodes 160a and 160b are placed on opposite sides of the target site 212 to contact different portions of skin of a user to apply a current in a manner so as to provide electronic anesthesia. The specific placement of the electrodes 160a and 160b is not particularly limited, but in some implementations, the electrodes 160a and 160b may be placed apart such that target site 212 of injection or piercing by the needle 210 is disposed between the electrodes 160a and 160b as illustrated. The transformer 170 (or any other device capable of increasing voltage that may be apparent to a person ordinary skill in the art) may be connected to one of the electrodes 160a by one of the electrical conductors 190a. As used herein, “connected” may include being coupled electronically, coupled communicatively, or both, in either a wireless or wired manner. The transformer 170 may also be connected to the power transistor 175 by one of the electrical conductors 190b. Another electrical conductor 190c may connect the power transistor 175 (or any other device capable of increasing current that may be apparent to a person of ordinary skill in the art) to the processor 185, which is connected to the other electrode 160b by another electrical conductor 190e. The electrical conductor 190e may also connect the power source 145 to both the other electrode 160b and the processor 185.

The processor 185 is not particularly limited and may include a microprocessor or any other computer processor that may be apparent to a person of ordinary skill in the art. The processor 185 may control the power transistor 175 and the transformer 170 to supply electricity from the power source 145 to the electrodes 160a and 160b to apply electronic anesthesia to the user's skin. By using electronic anesthesia controlled by the processor 185, the medical device 100 may block pain sensation prior to, during, and after injection or piercing of the target site 212 by the needle 210 through transcutaneous electrical nerve stimulation. In some implementations, the processor 185 may control the power transistor 175 and the transformer 170 to supply a micro-current (e.g. 25 to 900 micro-amps) to the electrodes 160a and 160b. The micro-current may be a single-phase alternating current (AC) carrier signal (for example, 10,000 to 19,000 Hz) that may be modulated on and off over time (e.g., at between 0.3 Hz up to 10,000 Hz) to block pain sensation in a user's skin. Further, in some example implementations, the AC carrier signal may be further inverted every second by reversing the polarity of the signal between the electrodes 160a and 160b. Of course, other micro-current values may be selected to facilitate pain blocking in the user as may be apparent to a person of ordinary skill in the art.

The processor 185 is also connected to an amplifier 165 by another electrical conductor 190f. The amplifier 165 is connected to the diagnostic chip 140 by another electrical conductor 190g. The diagnostic chip 140 is also connected directly to the processor 185 by the electrical conductor 190h. The processor 185 may control the diagnostic chip 140 to analyze the fluids transported to the fluid collection chamber 135 in the capillary collection tube 180. The analysis by the diagnostic chip 140 is discussed in greater detail below.

The processor 185 may also be connected to the indicator 130 by the electrical conductor 190d. Based on the analysis by the diagnostic chip 140, the processor 185 may control the indicator 130 to provide information to a user of the medical device 100. The indicator 130 may provide status information (such as “ready for testing,” “testing complete,” etc.), diagnostic information (such as “test positive,” “test negative,” etc.) or any other information that may be apparent to a person of ordinary skill in the art. The indicator 130 may be a light source (such as a light bulb, an LED, etc.), an audio indicator (such as a speaker, etc.), or any other type of indicator that may be apparent to a person ordinary skill in the art.

Further, in some implementations the wearable housing 105 may also include a wireless communication module 195 connected to the processor 185 by an electrical conductor 190i. The wireless communication module 195 may include a transceiver configured to communicate via Bluetooth, Wi-Fi, cellular, radio or any other wireless communication technology that may be apparent to a person of ordinary skill in the art. The wireless communication module 195 may be used by the processor 185 to communicate the analysis results of the diagnostic chip 140 to a computing device (Not illustrated in FIG. 2). In some example implementations, the wireless communication module 195 may be replaced or supplemented with a wired communication module configured to communicate analysis results of the diagnostic chip 140 to a computing device via a wired communications link.

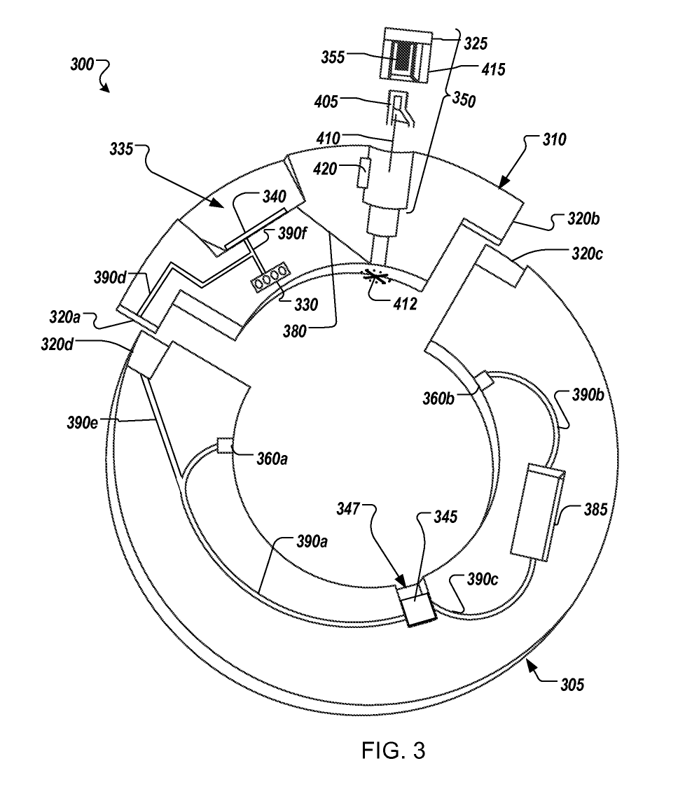

FIG. 3 illustrates an exploded view of another example implementation of the medical device 300 illustrated in FIG. 1. The medical device 300 may have components similar to the components illustrated in FIG. 2 and some components have been omitted for ease of visualization and discussion of components illustrated in FIG. 3. Further, some of the components of the medical device 100 illustrated in FIG. 2 may have been relocated in the medical device 300 of FIG. 3. For example, an indicator 330 has been relocated to the cartridge 310 and the power source 345 has been relocated to the wearable housing 305. The medical device 300 is formed by a wearable housing 305 and a cartridge 310 removably coupled to the wearable housing 305. The cartridge 310 is coupled to the wearable housing 305 by connectors 320a, 320b, 320c, and 320d. Specifically, a connector 320a on the cartridge 310 engages a connector 320d on the wearable housing 305. Further, a connector 320b on the cartridge 310 engages a connector 320c on the wearable housing 305. The connectors 320a, 320b, 320c, and 320d may provide one or more of: (1) a physical connection to secure the cartridge 310 to the wearable housing 305, (2) an electrical connection to allow transmission of electrical current (such as direct current (DC) or alternating current (AC)) between the wearable housing 305 and the cartridge 310, and (3) a communication connection to allow transmission of signals between the wearable housing 305 and the cartridge 310. The connectors 320a, 320b, 320c, and 320d are not particular limited but may include snap connectors, press-fit connectors, magnetic connectors, or any other type of connector that may be apparent to a person of ordinary skill in the art.

In the example implementation of FIG. 3, the cartridge 310 may include the fluid collection chamber 335, a diagnostic chip 340, the indicator 330, a retractable needle mechanism 350, and a capillary collection tube 180. The wearable housing 305 is illustrated as including a pair of electrodes 360a and 360b, a processor 185, the power source 345 and a plurality of electrical conductors 190a-190h interconnecting the components. In some implementations, the power source 345 may be a battery, a fuel cell, or any other power source that may be apparent to a person of ordinary skill in the art.

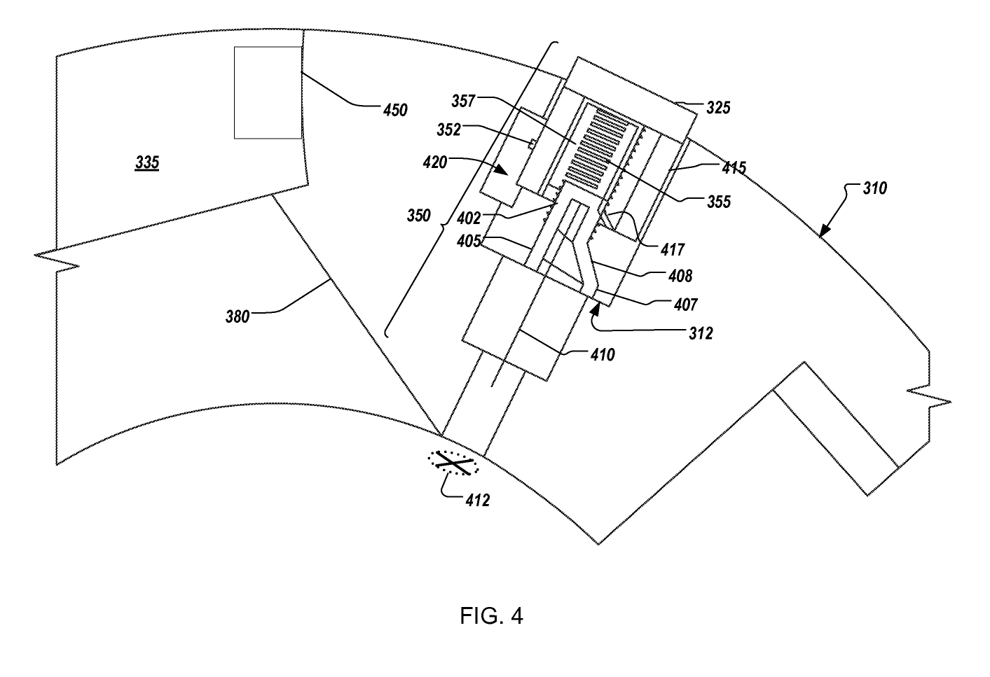

In the cartridge 310, the retractable needle mechanism 350 includes the triggering mechanism 325, an injector housing 415, a biasing member 355, a needle 410 and a needle housing 405. The triggering mechanism 325 may be attached to an upper exterior surface of the injector housing 415. The biasing member 355 may be located within the injector housing 415 and above the needle housing 405. The needle housing 405 may be located above and may surround an upper end of the needle 410. A retraction groove 420 may be located adjacent the needle housing 405. The retractable needle mechanism 350 is configured to transmit downward movement of the triggering mechanism 325 into downward movement of the needle 410. The interaction of the triggering mechanism 325 and the retractable needle mechanism 350 is discussed in greater detail below with respect to the FIG. 4. The needle 410 is designed to pierce the skin of a user wearing the medical device 300 to release fluid (such as blood cells, lymph fluid, or any other fluid that may be apparent to a person of ordinary skill in the art). For reference, an area directly below the needle 410 (referred to herein as a target site 412) is illustrated to show a general area where the needle 410 may pierce the skin of the user wearing the medical device 300 to release the fluid.

As illustrated, the capillary collection tube 380 is positioned and configured to collect the released fluid from the target site 412 and transport the fluid into the fluid collection chamber 335 wherein it is analyzed using the diagnostic chip 340. As discussed in greater detail below, the diagnostic chip 340 may use lab-on-a-chip techniques to analyze the collected fluid to detect one or more chemicals, antigens, or proteins found in the sample. The connectors 320a, 320b, 320c, and 320d may provide an electrical connection between the cartridge 310 and the wearable housing 305 to allow the power source 345 to power the diagnostic chip 340 via the electrical conductors 390d and 390e.

Further, in some implementations, the cartridge 310 may also include an indicator 330 electrically connected to the diagnostic chip 340 by an electrical conductor 390f. Based on the analysis by the diagnostic chip 140, the indicator 330 to provide information to a user of the medical device 300. The indicator 330 may provide status information (such as “ready for testing,” “testing complete,” etc.), diagnostic information (such as “test positive,” “test negative,” etc.) or any other information that may be apparent to a person of ordinary skill in the art. The indicator 330 may be a light source (such as a light bulb, an LED, etc.), an audio indicator (such as a speaker, etc.), or any other type of indicator that may be apparent to a person ordinary skill in the art.

As discussed above, the wearable housing 305 may provide the pair of electrodes 360a and 360b, which are placed on opposite sides of the target site 412 to contact different portions of skin of a user to apply a current for electronic anesthesia. Again, the specific placement of the electrodes 360a and 360b is not particularly limited, but in some implementations, the electrodes 360a and 360b may be placed apart such that target site 412 of injection or piercing by the needle 410 is disposed between the electrodes 360a and 360b as illustrated. In FIG. 3, a simplified electrical system is shown within the wearable housing 305. Specifically, the electrical system includes the power source 345 installed in power terminal 347. The power terminal 347 may connect the power source 345 to electrical conductors 390a and 390c. The electrical conductor 390a may connect the power terminal 347 to one of the electrodes 360a and electrical conductor 390e discussed above. The electrical conductor 390c may connect the power terminal 347 to the processor 385. Further, another electrical conductor 390b may connect the processor 385 to the other electrode 360b. The electrical system of the medical device 300 is not limited to this simplified structure and may include any additional components that may be apparent to a person of ordinary skill in the art. For example the electrical system of the medical device 300 may also include a transformer, power transistor, an amplifier, or any other circuit components that may be apparent to a person of ordinary skill in the art.

The processor 385 may supply electricity from the power source 345 to the electrodes 360a and 360b to apply electronic anesthesia to the user's skin. By using electronic anesthesia controlled by the processor 385, the medical device 300 may block pain sensation prior to, during, and after injection or piercing of the target site 412 by the needle 410 through transcutaneous electrical nerve stimulation. In some implementations, the processor 385 may supply a micro-current (e.g. 25 to 900 micro-amps) to the electrodes 160a and 160b. The micro-current may be a single-phase alternating current (AC) carrier signal (for example, 10,000 to 19,000 Hz) may be modulated on and off over time (e.g., at between 0.3 Hz up to 10,000 Hz) to block pain sensation in a user's skin. Further, in some implementations, the AC carrier signal may be further inverted every second by reversing the polarity of the signal between the electrodes 360a and 360b. Of course, other micro-current values may be selected to facilitate pain blocking in the user as may be apparent to a person of ordinary skill in the art.

In some implementations, the processor 385 may also be connected to the diagnostic chip 340 and control the diagnostic chip 340 to analyze the fluids transported to the fluid collection chamber 335. Further, in some implementations the wearable housing 305 may also include a wireless communication module (not illustrated) connected to the processor 385. Such a wireless communication module may include a transceiver configured to communicate via Bluetooth, Wi-Fi, cellular, radio or any other wireless communication technology that may be apparent to a person of ordinary skill in the art. The wireless communication module may be used by the processor 385 to communicate the analysis results of the diagnostic chip 340 to a computing device (not illustrated in FIG. 3). In some example implementations, the wireless communication module may be replaced or supplemented with a wired communication module configured to communicate analysis results of the diagnostic chip 340 to a computing device via a wire communications link.

FIG. 4 illustrates an enlarged, internal view of a retractable needle mechanism 350 of an example implementation of the cartridge 310 illustrated in FIG. 3. Though illustrated in the example implementation of the cartridge 310 of FIG. 3, the structure of the retractable needle mechanism 350 is not limited to this implementation and may be used in other implementations that may be apparent to a person of ordinary skill in the art. As illustrated, the retractable needle mechanism 350 includes the triggering mechanism 325, the injector housing 415, a biasing member 355, a needle 410, and a needle housing 405. The triggering mechanism 325 may be attached to an upper exterior surface of the injector housing 415. The injector housing 415 may have a hollow interior region 357 and a tab 352 on an exterior side surface. The hollow interior region 357 may include an angled portion 417 located at a lower end of the injector housing 415. The biasing member 355 may be positioned within the hollow interior region 357.

The needle housing 405 may be located below injector housing 415. The needle housing 405 includes an upper portion 402 and a lower portion 407. The upper portion 402 of the needle housing 405 may have a smaller width than the lower portion 407 of the needle housing 405. The needle housing 405 may also have an angled transition region 408 located between the upper portion 402 and the lower portion 407. The upper portion 402 of the needle housing 405 may be configured to fit within the hollow interior region 357 of the injector housing with the biasing member 355 being positioned between the injector housing 415 and the needle housing 405. The needle 410 may be attached to an underside of the lower portion 407 of the needle housing 405. The lower portion 407 of the needle housing 405 rests on a ledge 312 formed on the interior of the cartridge 310.

The needle 410 may be attached to an underside of the lower portion 407 of the needle housing 405. The needle 410 is designed to pierce the skin of a user wearing the medical device 300 to release fluid (such as blood cells, lymph fluid, or any other fluid that may be apparent to a person of ordinary skill in the art). For reference, an area directly below the needle 410 (referred to herein as a target site 412) is illustrated to show a general area where the needle 410 may pierce the skin of the user wearing the medical device 300 to release the fluid.

When a pressure is applied to the triggering mechanism 325 by a user, the injector housing 415 may be moved downward toward the needle housing 405. As the injector housing 415 moves downward, the ledge 312 may lock the injector housing 415 in position and may prevent downward movement of the needle housing 405 causing the injector housing 415 to move relative to the needle housing 405. This ledge 312 may allow the retractable needle mechanism 150 to resist downward movement of the triggering mechanism 325 until the downward movement of the triggering mechanism 325 exceeds a threshold distance. As the injector housing 415 moves relative to the needle housing 405, the upper portion 402 of the needle housing 405 may become increasingly inserted into the hollow interior region 357 of the injector housing 415. As the needle housing 405 is increasingly inserted into the hollow interior region 357, the biasing member 355 may become increasingly compressed. As the biasing member 355 compresses, the biasing member 355 may apply an increasing downward biasing force to the needle housing 405. As the upper portion 402 of the needle housing 405 is inserted into the hollow interior region 357 of the injector housing 415, the angled transition region 408 of the needle housing 405 may contact the angled portion 417 located at the lower end of the injector housing 415.

Once the angled transition region 408 of the needle housing 405 contacts the angled portion 417 of the injector housing 415, further downward movement of the injector housing 415 may cause the needle housing 405 to move orthogonally along the ledge 312 until the lower portion 407 of the needle housing 405 is no longer resting on the ledge 312. In other words, when the downward movement of the triggering mechanism 325 exceeds a threshold distance, further downward movement of the injector housing 415 causes the needle housing 405 to move relative to the ledge 312. Once the lower portion 407 of the needle housing 405 is no longer resting on the ledge 312, the biasing force applied by the biasing member 355 may cause the needle housing 405 and the needle 410 to rapidly move downward toward the target site 412, piercing the user's skin, and releasing the fluid. Capillary action may cause the released fluid to automatically move up the capillary collection tube 380 to the fluid collection chamber 335 for analysis.

After the needle 410 has pierced the user at the target site 412, the needle 410 and needle housing 405 retract into the retraction groove 420 and may be retained in a retracted position by the tab 352, which may ensure that a user cannot be pierced by the needle 410 more than one time (i.e. the retractable needle mechanism 350 may be prevented from further ejection and become disabled (e.g. a one-time use needle mechanism)). Further, in some implementations the cartridge 310 may also include a sterilization mechanism 450 to sterilize the fluid collection chamber 335 and capillary collection tube 380 from any fluids after analysis. For example, sterilization mechanism 450 may be a heating element configured to provide heat or thermal energy to the cartridge 310 to kill any bacteria, or viruses and denature any contaminants such as proteins or other bodily tissues present. The thermal energy may also be transmitted to the wearable housing 305 (illustrated in FIG. 3) to also sterilize the wearable housing 305. In another example implementation, the sterilization mechanism 450 may be a breakable or frangible housing (e.g. a glass or plastic housing designed to rupture or dissolve under specific or controlled circumstances as may be apparent to a person of ordinary skill in the art) containing a caustic solution such as an acid solution, a basic solution, topical anti-biotic, topical anti-viral, or any other solution that may be apparent to a person of ordinary skill in the art to sterilize the cartridge 310 and/or wearable housing 305 (illustrated in FIG. 3).

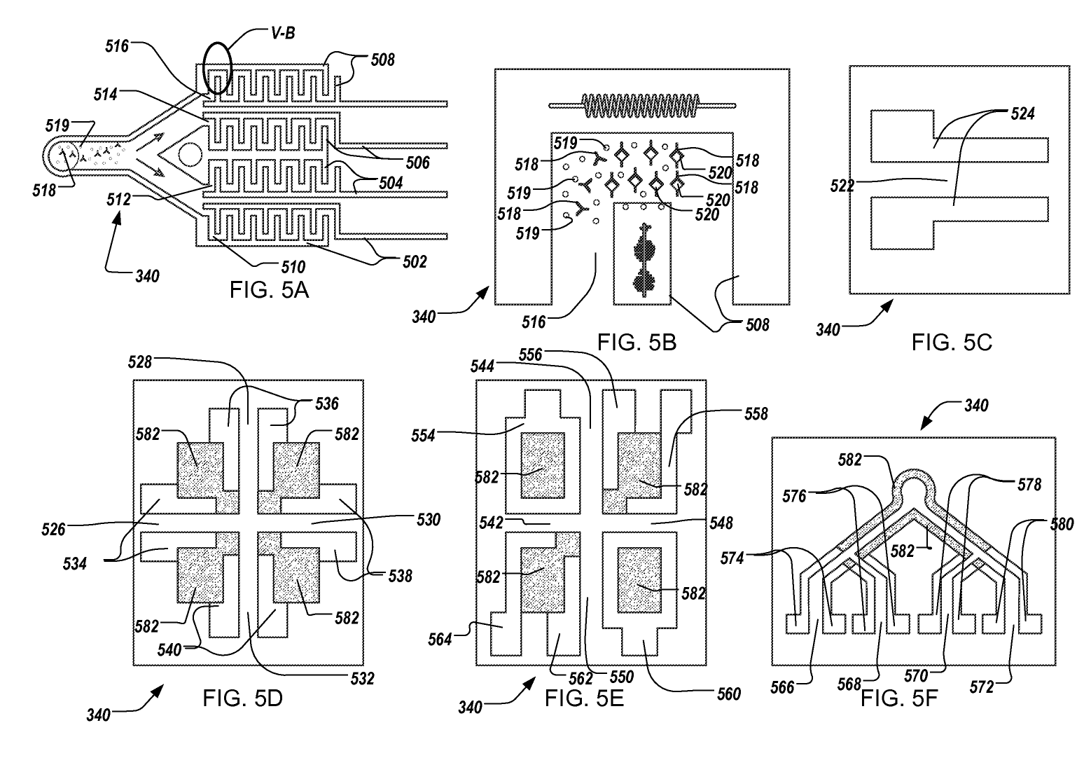

FIGS. 5A-5F illustrate example implementations of a diagnostic chip 340 used in an example implementation of the medical device 300 illustrated in FIG. 3. Though illustrated in the example implementation of the diagnostic chip 340 of FIG. 3, the structure of the diagnostic chip 340 is not limited to this implementation and may be used in other implementations that may be apparent to a person of ordinary skill in the art.

As may be understood by a person of ordinary skill in the art, a user may naturally produce specific proteins (e.g., antibodies Abs) as an immunological response to the presence of any foreign substances (e.g., antigens (Ags)). Each Ab may have a unique structure recognizable by a corresponding Ag via a lock-and-key mechanism. Many immunoassays are based on the sensitivity and specificity of this Ab-Ag interaction. Many current microfluidic immunoassay (e.g., micro-assay) systems permit miniaturization, integration, and automation.

In some implementations, the diagnostic chip 340 may use the microfluidic immunoassay systems to analyze the collected fluids. FIG. 5A illustrates one example implementation of a diagnostic chip 340. In FIG. 5A, the diagnostic chip 340 is formed with four pairs of electrodes 502, 504, 506, and 508, each targeting specific antibodies associated with antigens of a specific disease or condition. For example, each pair of electrodes 502, 504, 506, and 508 may target antibodies associated with a different sexually transmitted disease (such as syphilis, gonorrhea, trichomoniasis, chlamydia, or any other disease that may be apparent to a person of ordinary skill in the art.) When a fluid is added to the diagnostic chip 340, the fluid may include various different types of antigens, chemicals, or proteins (illustrated as antigens 518 and 519).

In some implementations the pairs of electrodes 502, 504, 506, and 508 may be fabricated by jet printing hydrophobic material with a given pattern to promote the flow of the fluid sample within hydrophilic microfluidic channels 510, 512, 514, and 516 on a paper substrate between the pairs of electrodes 502, 504, 506, and 508. Using hydrophobic material printed on hydrophilic paper may allow driving fluid displacement without any required pumps for the fluid displacement. This may allow less sample/reagent consumption, reduced risk of contamination, high sensitivity, less unit cost, and a higher reliability and functionality. In some example implementations, each pairs of electrodes (502, 504, 506, and 508) may be replaced with a triad of electrodes. Further, in some example implementations, the paper substrate may be replaced with a plastic or ceramic substrate.

FIG. 5B illustrates an enlarged view of the region illustrated in FIG. 5A. With reference to FIG. 5B, the various antigens, chemicals, or proteins (illustrated as antigens 518 and 519) from the collected fluid may flow through the hydrophilic microfluidic channel 516. A plurality of antibodies, chemicals, or proteins 520 may be bound to one of the electrodes 508. If the fluid flowing through the hydrophilic microfluidic channel 516 contains antigens, chemicals, or proteins having a structure recognizable (e.g., 518) to the plurality of antibodies, chemicals, or proteins 520 bound to the electrode 508, the antigens, chemicals, or proteins 518 will bind to the electrode 508. Conversely, antigens, chemicals, or proteins not having a recognizable structure (e.g., 519) will not bind to the electrode 508. As the antigens, chemicals, or proteins 518 will bind to the electrode 508, the impedance between the pair of electrodes 508 will change. By detecting or reading the impedance change with a sensor or microprocessor, the antigens, chemicals, or proteins 518 in the fluid may be identified. In other words, detected impedance changes may correspond to specific binding of antibodies with antigens and converts biological responses into electronic signals through electrochemical reaction that allows parallel and sensitive detection.

In some implementations, the diagnostic chip 340 may be formed from a nitrocellulose paper that may be patterned with the pairs of electrodes (502, 504, 506, and 508) and hydrophilic microfluidic channels (510, 512, 514, and 516) to generate multiple test zones. Each line of the diagnostic chip 340 could serve as a detection zone and prevent from cross contamination. This patterned structure may allow manipulation of fluids for complex and multiple analyses.

Example implementations of the diagnostic chip 340 may only require a few microliters per detection zone to detect the presence of the targeted antigens, chemicals, or proteins 518.

Example implementations of the diagnostic chip 340 are not limited to the layout of immunoassay electrode pairs (502, 504, 506, and 508) and hydrophilic microfluidic channels (510, 512, 514, and 516) illustrated in FIGS. 5A and 5B. FIGS. 5C-5F illustrate other example implementations of layouts of the diagnostic chip 340. FIG. 5C includes a single hydrophilic microfluidic channel 522 between a single pair of electrodes 524. FIG. 5D illustrates a design that may reduce space with geometrical optimization by arranging four channels (526, 528, 530, and 532) equally distributed in a radial array with four pairs of electrodes (534, 536, 538, and 540). FIG. 5E illustrates a design having features of the radial array with four channels (542, 544, 548, and 550) in a six-electrode (554, 556, 558, 560, 562, and 564) configuration. In this configuration, electrodes 554 and 560 represent common reference electrodes provided to optimize space and minimize the number of electrodes. Other implementations try to reduce space, and provide the adequate electrical conditions for the proper signal acquisition. FIG. 5F illustrates a branched array that includes four channels (566, 568, 570, and 572) with a pair of electrodes (574, 576, 578, and 580) provided for each channel (566, 568, 570, and 572). These example implementations of the diagnostic chip 340 may also include one or more hydrophobic regions that will conduct the fluid flow into the different channels (566, 568, 570, and 572).

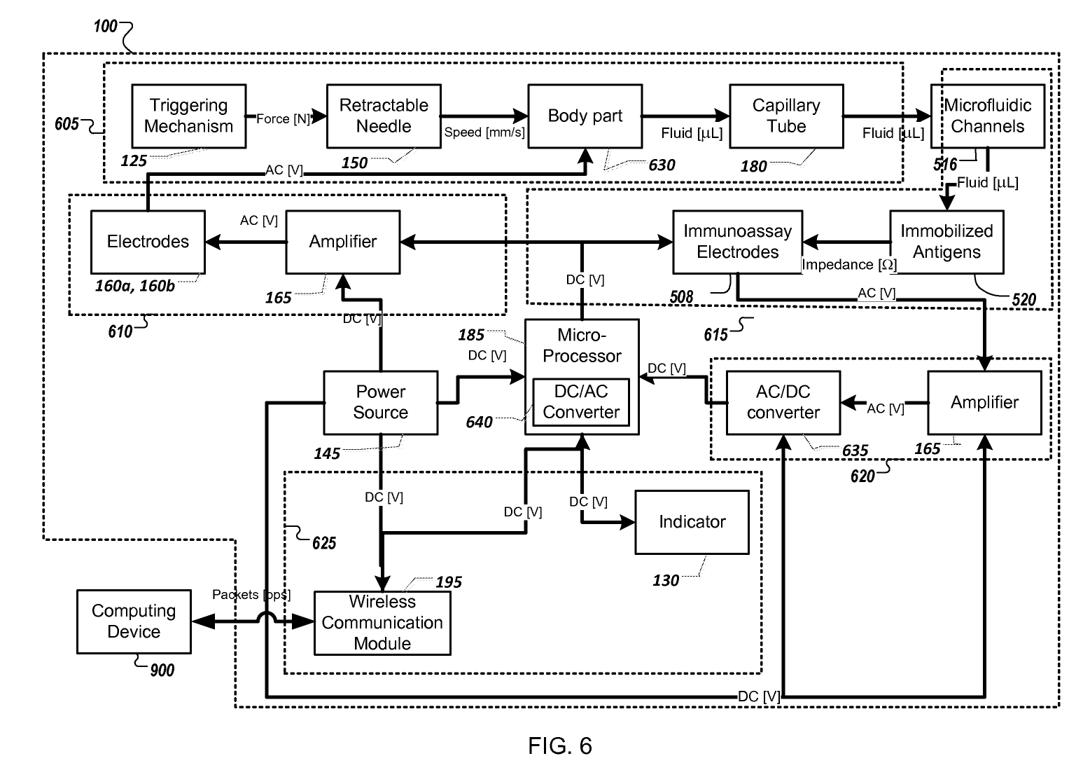

FIG. 6 illustrates an operational diagram of the medical device 100 illustrated in FIGS. 1 and 2. Operationally, the medical device 100 is formed by 5 subsystems: i) the fluid collection system 605, ii) the electronic anesthesia system 610, iii) the fluid analysis system 615, iv) the signal processor 620, and v) the communications module 625. The medical device 100 also includes the power source 145 and the processor 185, which control and power the electronic anesthesia system 610, fluid analysis system 615, the signal processor 620, and the communications module 625.

The fluid collection system 605 may include the triggering mechanism 125, the retractable needle mechanism 150, and the capillary collection tube 180. The operation of the fluid collection system 605 may be initiated when a user presses the triggering mechanism 125. As discussed above, the triggering mechanism 125 may be a button or other triggering structure that may be apparent to a person of ordinary skill in the art. When the triggering mechanism 125 is pressed, the triggering mechanism 125 activates the retractable needle mechanism 150.

As discussed above, with respect to FIG. 4, the structure of the retractable needle mechanism 150 may resist downward movement until the downward movement of the triggering mechanism 125 exceeds a threshold distance. When the threshold distance is exceeded, retractable needle mechanism 150 releases and a biasing member (155 in FIG. 2) applies a force to a needle (210 in FIG. 2), resulting in the acceleration of the needle (210) in FIG. 2, which hits the body part 630 (shown in FIG. 6) of the user and releases an amount of fluid. The body part 630 (shown in FIG. 6) may be a thumb, finger, toe, or other body part that might be apparent to a person of ordinary skill in the art. Due to the structure of the retractable needle mechanism 150, the needle 210 may be prevented from further ejection and become disabled. The released amount of fluid may be drawn into the capillary collection tube 180 where it is transported to the fluid analysis system 615.

The electronic anesthesia system 610 may include the electrodes 160a, 160b, and the amplifier 165. Further, internal to the microprocessor 185, a direct current (DC) to AC (DC/AC) converter 640 may be provided to allow the microprocessor 185 to produce an alternating pulse. In some example implementations, the DC/AC converter 640 may be omitted and the microprocessor 185 may send a pulse signal or the microprocessor 185 may be omitted and instead a timer (e.g., an LM555 timer) may be used. During the above discussed operation of the fluid collection system 605, the electronic anesthesia system 610 may apply a current to the body part 630 (shown in FIG. 6) to create a localized numbing effect during fluid collection. Specifically, the body part 630 (shown in FIG. 6) may contact the two electrodes 160a, 160b. The electrodes 160a, 160b may be connected to the amplifier 165, which may be connected to the DC/AC converter 640 and the power source 145. The DC/AC converter 640 may also be connected to the power source 145 and the processor 185. The processor 185 may generate an electric pulse that is mounted on a DC carrier signal from the power source 145 as on offset. The amplifier 165 may then amplify the offset pulse to apply a controlled alternating micro-current to the electrodes 160a, 160b, which contact the body part 630 (shown in FIG. 6).

In other words, the processor 185 controls the power source 145 to generate, condition, and amplify a micro-current. The electrodes 160a, 160b pass the micro-current through the body part 630 (shown in FIG. 6), blocking the sensation of pain. In some implementations, the electronic anesthesia system 610 may supply a micro-current (e.g. 25 to 900 micro-amps) to the body part 630 (shown in FIG. 6). The micro-current may be a single-phase alternating current (AC) carrier signal (for example, 10,000 to 19,000 Hz) may be modulated on and off over time (e.g., at between 0.3 Hz up to 10,000 Hz) to block pain sensation in a user's skin. Of course, other micro-current values may be selected to facilitate pain blocking in the user as may be apparent to a person of ordinary skill in the art.

The fluid analysis system 615 may include the hydrophilic microfluidic channels 516 and the immunoassay electrodes 508 of the diagnostic chip 340 (390 illustrated in FIGS. 5A and 5B) and the immobilized antigens, chemicals or proteins 520 attached to the immunoassay electrodes 508. The fluid analysis system 615 also receives a DC signal with an offset pulse from the microprocessor 185. When the collected fluid is transported to the fluid analysis system 615 (e.g., the diagnostic chip 140 of FIG. 1), the fluid is then split into a plurality of hydrophilic microfluidic channels 516 (such as those illustrated in FIGS. 5A and 5B). Within each of the hydrophilic microfluidic channels 516 a volume of immobilized antigens, chemicals, or proteins 520 may be attached to the immunoassay electrodes 508 on each side of the hydrophilic microfluidic channels 516. As discussed above, the hydrophilic microfluidic channels 516 may be manufactured by printing a conductive ink on a paper (such as a nitrocellulose paper). The conductive ink may serve as the electrodes 508 defining the hydrophilic microfluidic channels 516. In some example implementations, a wax may also be printed in order to create hydrophobic regions that will allow the flow of the fluid in a specific direction. In some example implementations, the electrodes may be formed from other conductive materials such as gold, copper or any other material that may be apparent to a person of ordinary skill in the art.

The processor 185 may be connected to immunoassay electrodes 508, via the DC/AC converter 640, to send an electrical signal to the immunoassay electrodes 508. The electrical signal from the processor 185 may experience changes of amplitude and/or phase due to changes in the electrical impedance that may be generated by an electrochemical reaction between the antigens, chemicals or proteins 520 attached to the immunoassay electrodes 508 and any antibodies, chemicals, or proteins present in the fluid as it flows through the hydrophilic microfluidic channels 516. The changes of the amplitude and/or phase of the electrical signal may be processed by the signal processor 620 as discussed below.

The signal processor 620 may include an AC to DC (AC/DC) converter 635 and an amplifier 165. In some implementations, the amplifier 165 may be shared with the electronic anesthesia system 610. In some implementations, separate amplifiers 165 may be used by the signal processor 620 and the electronic anesthesia system 610. As discussed above, when the collected fluid flows through the hydrophilic microfluidic channels 516, the electrochemical reaction may occur between the antigens, chemicals or proteins 520 attached to the immunoassay electrodes 508 and any antibodies, chemicals, or proteins present in the fluid. This electrochemical reaction can cause changes of the amplitude and/or phase of the electrical signal applied to the immunoassay electrodes 508 by the processor 185. The changes of the amplitude and/or phase of the electrical signal may be detected, and amplified by the amplifier 165. The output of the amplifier 165 may then be conditioned by the AC/DC converter 635 and sent as feedback to the processor 185. The processor 185 may monitor the feedback signal to determine the presence of antibodies, chemicals, or proteins in the fluid. Based on a determination of the presence of antibodies, chemicals, or proteins in the fluid, the processor 185 may control the communications module 625 to communicate the information relating to the determination.

The communications module 625 may include the indicator 130 and the wireless communication module 195. When the processor 185 determines that a specific antibody, chemical, or protein is present in the fluid, the processor 185 may control the indicator 130 to communicate information to the user. The indicator 130 may provide status information (such as “ready for testing,” “testing complete,” etc.), diagnostic information (such as “test positive,” “test negative,” etc.) or any other information that may be apparent to a person of ordinary skill in the art. The indicator 130 may be a light source (such as a light bulb, an LED, etc.), an audio indicator (such as a speaker, etc.), or any other type of indicator that may be apparent to a person ordinary skill in the art.

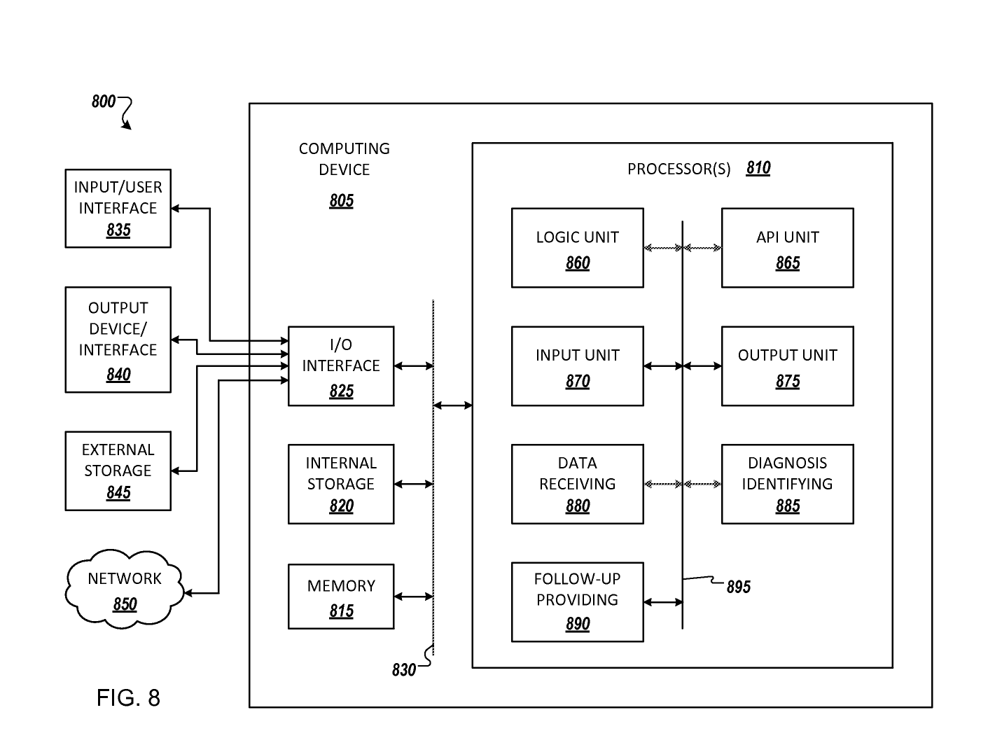

Further, when the processor 185 determines that a specific antibody, chemical, or protein is present in the fluid, the processor 185 may also control the wireless communication module 195 to transmit information to the computing device 805 (illustrated in greater detail in FIG. 8 discussed below).

The wireless communication module 195 may include a transceiver configured to communicate via Bluetooth, Wi-Fi, cellular, radio or any other wireless communication technology that may be apparent to a person of ordinary skill in the art. The wireless communication module 195 may include a transceiver configured to communicate via Bluetooth, Wi-Fi, cellular, radio or any other wireless communication technology that may be apparent to a person of ordinary skill in the art. In some example implementations, the wireless communication module 195 may be replaced or supplemented with a wired communication module configured to communicate analysis results to the computing device 805 via a wired communications link.

In some implementations, the wireless communication module 195 may establish a paired connection with the computing device 805 that is authenticated through an application installed on the computing device 805. The computing device 805 may present results of the analysis by the medical device 100 to the user on a screen of the mobile device, together with other relevant information.

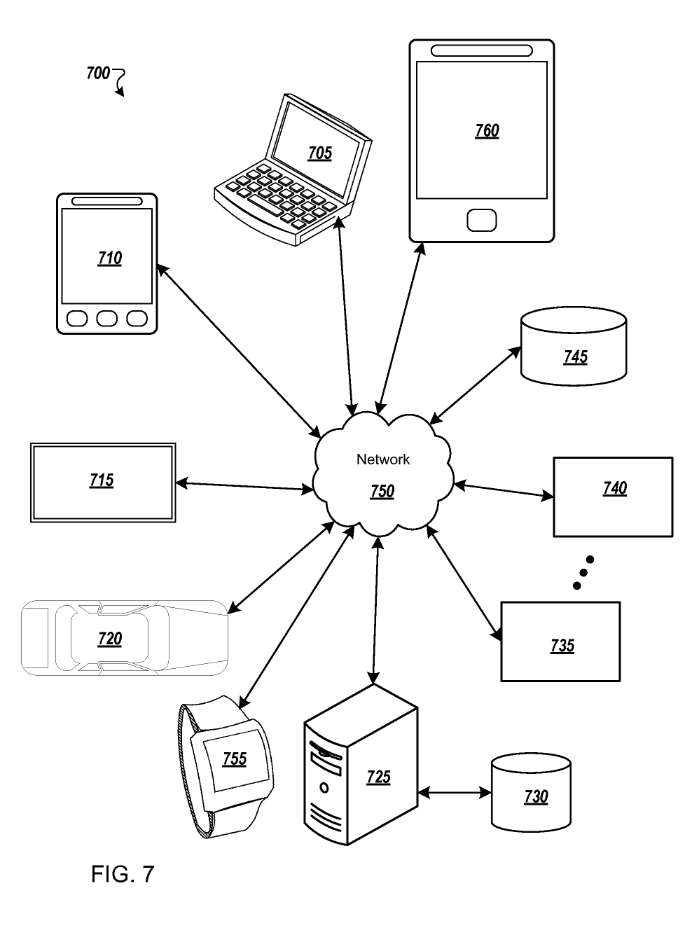

FIG. 7 shows an example environment 700 suitable for some example implementations. Environment 700 includes devices 705-745, and each is communicatively connected to at least one other device via, for example, network 760 (e.g., by wired and/or wireless connections). Some devices may be communicatively connected to one or more storage devices 730 and 745.

An example of one or more devices 705-745 may be computing device 805 described below in FIG. 8. Devices 705-745 may include, but are not limited to, a computer 705 (e.g., a laptop computing device), a mobile device 710 (e.g., smartphone or tablet), a television 715, a device associated with a vehicle 720, a server computer 725, computing devices 735-740, storage devices 730 and 745. Computing devices 760 illustrate an implementation as a tablet device.

Further computing device 755 also includes wearable computing devices (e.g. a smartwatch, smart ring, smart bracelet, etc.). In particular, the use of wearable computing devices 755 may provide additional functionality over tablets, phones, and other computing devices by directly monitoring patient vitals and other information by being attached directly to the patient. The wearable computing device 755 may also include an example implementation of the medical device 100 or the medical device 300 illustrated above. For example, the wearable computing device 755 may be considered the wearable housing 105, 305, and all components thereof discussed herein. Further, the wearable computing device 755 may be configured to receive a cartridge 110, 310 to allow collection and analysis of a fluid sample collected by a user wearing the wearable computing device 755.

FIG. 8 shows an example computing environment 800 with an example computing device 805 suitable for use in some example implementations. A computing device 805 in computing environment 800 can include one or more processing units, cores, or processors 810, memory 815 (e.g., RAM, ROM, and/or the like), internal storage 820 (e.g., magnetic, optical, solid state storage, and/or organic), and/or I/O interface 825, any of which can be coupled on a communication mechanism or bus 830 for communicating information or embedded in the computing device 805.

Computing device 805 can be communicatively coupled to input/user interface 835 and output device/interface 840. Either one or both of input/user interface 835 and output device/interface 840 can be a wired or wireless interface and can be detachable. Input/user interface 835 may include any device, component, sensor, or interface, physical or virtual, which can be used to provide input (e.g., voice, buttons, touch-screen interface, keyboard, a pointing/cursor control, microphone, camera, braille, motion sensor, optical reader, and/or the like). Output device/interface 840 may include a display, television, monitor, printer, speaker, braille, or the like. In some example implementations, input/user interface 835 and output device/interface 840 can be embedded with or physically coupled to the computing device 805. In other example implementations, other computing devices may function as or provide the functions of input/user interface 835 and output device/interface 840 for a computing device 805.

Examples of computing device 805 may include, but are not limited to, highly mobile devices (e.g., smartphones, devices in vehicles and other machines, devices carried by humans and animals, and the like), mobile devices (e.g., tablets, notebooks, laptops, personal computers, portable televisions, radios, and the like), and devices not designed for mobility (e.g., desktop computers, other computers, information kiosks, televisions with one or more processors embedded therein and/or coupled thereto, radios, and the like).

Computing device 805 can be communicatively coupled (e.g., via I/O interface 825) to external storage 845 and network 850 for communicating with any number of networked components, devices, and systems, including one or more computing devices of the same or different configuration. I/O interface 825 can include, but is not limited to, wired and/or wireless interfaces using any communication or I/O protocols or standards (e.g., Ethernet, 802.11x, Universal System Bus, WiMax, modem, a cellular network protocol, and the like) for communicating information to and/or from at least all the connected components, devices, and network in computing environment 800. The Network 850 may also be used to communicate with an example implementation of a medical device as described herein (e.g. medical device 100 and/or medical device 300). The Network 850 can be any network or combination of networks.

Computing device 805 can use and/or communicate using computer-usable or computer-readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CD ROM, digital video disks, Blu-ray disks), solid state media (e.g., RAM, ROM, flash memory, solid-state storage), and other non-volatile storage or memory.

Computing device 805 can be used to implement techniques, methods, applications, processes, or computer-executable instructions in some example computing environments. Computer-executable instructions can be retrieved from transitory media, and stored on and retrieved from non-transitory media. The executable instructions can originate from one or more of any programming, scripting, and machine languages (e.g., C, C++, C#, Java, Visual Basic, Python, Perl, JavaScript, and others).

Processors 810 can execute under any operating system (OS) (not shown), in a native or virtual environment. One or more applications can be deployed that include logic unit 860, application programming interface (API) unit 865, input unit 870, output unit 875, data receiving unit 880, diagnosis identifying unit 885, follow-up information providing unit 890, and inter-unit communication mechanism 895 for the different units to communicate with each other, with the OS, and with other applications (not shown). For example, data receiving unit 880, diagnosis identifying unit 885, follow-up information providing unit 890 may implement one or more of the processes disclosed herein. The described units and elements can be varied in design, function, configuration, or implementation and are not limited to the descriptions provided.

In some example implementations, when information or an execution instruction is received by API unit 865, it may be communicated to one or more other units (e.g., logic unit 860, input unit 870, output unit 875, data receiving unit 880, diagnosis identifying unit 885, and follow-up information providing unit 890). As explained below, data receiving unit 880 may be implemented to receive data representative of detection of an antigen, an antibody, chemical, or protein detected by a medical device (e.g. medical device 100 and/or medical device 300); the diagnosis identifying unit 885 may determine a diagnosis of a user based on the received data as explained below; and the follow-up information providing unit 890 may provide the user with follow-up information based on the determined diagnosis.

In some instances, logic unit 860 may be configured to control the information flow among the units and direct the services provided by API unit 865, input unit 870, output unit 875, data receiving unit 880, diagnosis identifying unit 885, and follow-up information providing unit 890 in some example implementations described above. For example, the flow of one or more processes or implementations may be controlled by logic unit 860 alone or in conjunction with API unit 865.

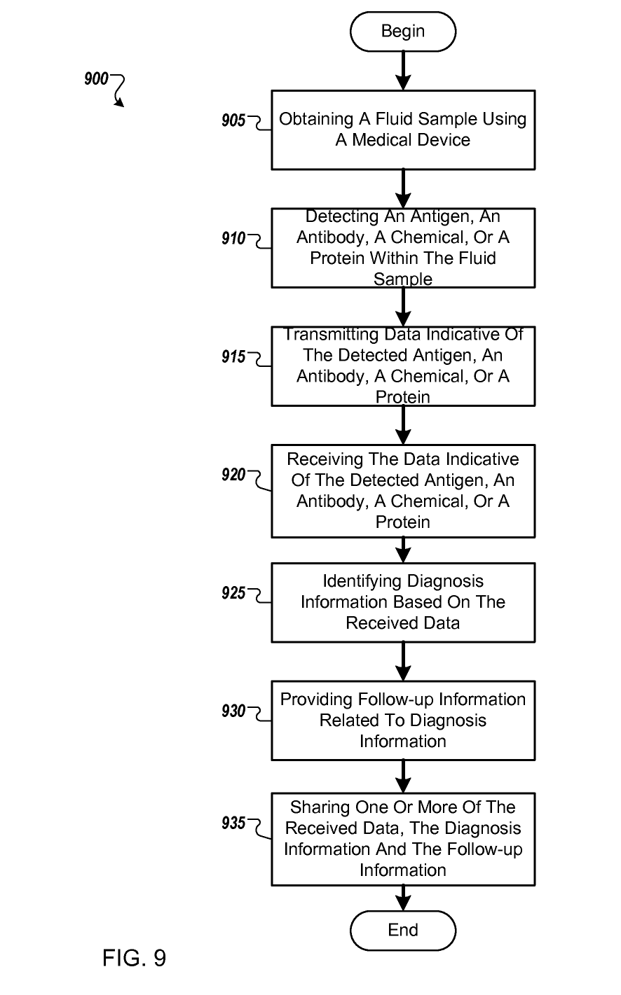

FIG. 9 illustrates a flowchart of a diagnostic method 900 according to an example implementation of the present application. As illustrated, the diagnostic method 900 includes obtaining a fluid sample using a medical device (such as the medical device 100 illustrated in FIGS. 1 and 2 and/or the medical device 300 illustrated in FIGS. 3 and 4) to obtain a fluid sample from a user at 905. The fluid sample collection may include piercing the user's skin using the retractable needle mechanism 150/350, collecting the fluid, and transporting the fluid via a capillary collection tube 180/380 to a fluid collection chamber 135/335 for analysis. The collected fluid may be blood, lymph fluid, or any other fluid that may be apparent to person of ordinary skill in the art for analysis and/or diagnostics.

After the fluid has been collected, the fluid may be analyzed for the presence of an antigen, an antibody, a chemical, or a protein, which may be detected in the fluid sample in 910. Within the fluid, any antigen, antibody, chemical, or protein indicative of any known bacteria, virus, disease, or condition may be detected. For example, antigens or antibodies associated with STDs (such as syphilis, gonorrhea, trichomoniasis, and/or chlamydia) may be detected. Other diseases or conditions may also be detected as may be apparent to a person of ordinary skill in the art. The detection may be performed using a diagnostic chip (such as the diagnostic chip 140 and/or the diagnostic chip 340 illustrated in FIGS. 1-4). As discussed above, the detected antigen, antibody, chemical, or protein may cause an electrical impedance change in immunoassay electrodes and a processor may detect the electrical impedance change.

After, an antigen, antibody, chemical or protein is detected in the fluid sample, the medical device 100 and/or 300 may transmit data indicative of the detected antigen, antibody, chemical or protein to a computing device (such as the computing device 805 illustrated in FIG. 8) in 915. In some example implementations, the transmission of the data may be done using a wireless communication module of the medical device 100 and/or 300 (e.g. wireless communication module 195). The wireless communication module 195 may be configured to communicate with the computing device via Bluetooth, WI-Fi, cellular, radio or any other wireless communication technology that may be apparent to a person of ordinary skill in the art. In other example implementations, a wired communication module may be used to transmit the data to a computing device 805 via a wired communications link such as a serial connection, parallel port connection, USB connection, Ethernet connection, or any other wired connection that may be apparent to a person of ordinary skill in the art.

The computing device 805 may receive the transmitted data indicative of the detected antigen, antibody, chemical, or protein in 920. The computing device 805 may receive the transmitted data via wired or wireless connection. For example, the transmitted data may be received via Bluetooth, WI-Fi, cellular, radio or any other wireless communication technology or via serial connection, parallel port connection, USB connection, Ethernet connection, or any other wired connection.

Based on the received data indicative of the detected antigen, antibody, chemical, or protein, the computing device 805 may identify diagnosis information and provide the information to a user at 925. For example, if the data indicates that an antigen or antibody associated with a known STD (such as syphilis, gonorrhea, trichomoniasis and/or chlamydia) was detected in the fluid, the computing device 805 may identify diagnosis information indicative of the associate STD and inform the user that they may likely have the associated STD.

Though example implementations of the present application have been discussed in the context of STDs (such as syphilis, gonorrhea, trichomoniasis and/or chlamydia), example implementations are not limited to STDs. Example implementations of the present application may also be used to diagnose other diseases or conditions based on detection of known associated antigens, antibodies, chemicals or proteins, which may be detected in extracted fluids as may be apparent to a person of ordinary skill in the art. Additionally, example implementations of the present application are also not limited to diagnosis only and may also be adapted for treatment diseases or other conditions. For example, example implementations of the present application may be adapted to administer medicine, vaccines, or other compounds via injection through a user's skin using the retractable needle mechanism 150 and 350, as may be apparent to a person of ordinary skill in the art.

The computing device 805 may also provide follow-up information relating to the identified diagnosis information to the user at 930. For example, the follow-up information may include information regarding treatments of the diagnosed disease or condition. Further, the follow-up information may also include information on symptoms or complications of the diagnosed disease or condition. Further, in some implementations, the follow-up information may also include contact information (such as name, address, phone number, fax number, email address, website link, social media link, etc.) of a medical professional, clinic, or other provider that could provide treatment for the diagnosed disease or condition. The diagnosis information and the follow-up information may be presented to the user in a simplified, easy to navigate format.

The computing device 805 may also provide the user with options to share one or more of the received data indicative of the detected antigen, antibody, chemical or protein, the diagnosis information, and the follow-up information with third parties. For example, the computing device 805 may allow the user request to share the data, diagnosis information, and/or follow-up information with third parties via email, SMS message, website posting, social media posting or any other mechanism that may be apparent to a person of ordinary skill in the art. Further, the computing device 805 may also allow the user to share the data, diagnosis information, and/or follow-up information by uploading to an electronic medical record database or other database of medical information accessible by the user's medical caregivers.

In some implementations, the received data indicative of the detected antigen, antibody, chemical or protein, the diagnosis information, and/or the follow-up information may also be stored locally on the computing device 805. In some implementations, the computing device may be configured to automatically delete one or more of the received data indicative of the detected antigen, antibody, chemical or protein, the diagnosis information, and/or the follow-up information after the expiration of a certain amount of time. In some example implementations, the computing device may also be configured to encrypt the stored received data indicative of the detected antigen, antibody, chemical or protein, the diagnosis information, and/or the follow-up information. Encryption may be tied to one or more independent pins set by the user. Further, in some implementations, the computing device 805 may be configured to delete all received data, the diagnosis information, and/or the follow-up information stored locally by a very simple user operation.

Example implementations of the present application may also fully comply with any local, regional, national, or international laws governing the sharing, disclosure, encryption, protection, and storage of medical, psychological, social, physical, mental or personal information. Further, in example implementations, any data transmitted via wireless communication or by wired communication may be transmitted in an encrypted or anonymized manner to protect a user's information in full compliance with all local, regional, national, or international law.

The foregoing detailed description has set forth various example implementations of the devices and/or processes via the use of block diagrams, schematics, and examples. Insofar as such block diagrams, schematics, and examples contain one or more functions and/or operations, each function and/or operation within such block diagrams, flowcharts, or examples can be implemented, individually and/or collectively, by a wide range of hardware, software, firmware, or virtually any combination thereof. In one example implementation, the present subject matter may be implemented via Application Specific Integrated Circuits (ASICs). However, the example implementations disclosed herein, in whole or in part, can be equivalently implemented in standard integrated circuits, as one or more programs executed by one or more processors, as one or more programs executed by one or more controllers (e.g., microcontrollers), as firmware, or as virtually any combination thereof.

While certain example implementations have been described, these example implementations have been presented by way of example only, and are not intended to limit the scope of the protection. Indeed, the novel methods and apparatuses described herein may be embodied in a variety of other forms. Furthermore, various omissions, substitutions, and changes in the form of the methods and systems described herein may be made without departing from the spirit of the protection. The accompanying claims and their equivalents are intended to cover such forms or modifications as would fall within the scope and spirit of the protection.

Although a few example implementations have been shown and described, these example implementations are provided to convey the subject matter described herein to people who are familiar with this field. It should be understood that the subject matter described herein may be implemented in various forms without being limited to the described example implementations. The subject matter described herein can be practiced without those specifically defined or described matters or with other or different elements or matters not described. It will be appreciated by those familiar with this field that changes may be made in these example implementations without departing from the subject matter described herein as defined in the appended claims and their equivalents.

We claim:

1. A medical device comprising:
a housing; and
a diagnostic cartridge removably coupled to the housing, the diagnostic cartridge comprising:
a retractable needle mechanism disposed within the diagnostic cartridge, the retractable needle mechanism configured to extract a fluid sample;
a fluid collection chamber disposed the retractable needle mechanism, the fluid collection chamber configured to collect the fluid sample extracted by the retractable needle mechanism; and
a diagnostic chip disposed within the fluid collection chamber configured to analyze the fluid sample.

2. The medical device of claim 1, wherein the housing comprises:
a first electrode disposed on the housing, the first electrode configured to contact a first portion of a user;
a second electrode disposed on the housing a distance from the first electrode, the second electrode configured to contact a second portion of the user, and
a power source configured to provide a voltage between the first electrode and the second electrode to produce electronic anesthesia,
wherein the diagnostic cartridge is configured to removably couple to the housing between the first electrode and the second electrode.

3. The medical device of claim 1, wherein the housing has at least a partial ring structure configured to fit around a body part.

4. The medical device of claim 1, wherein a capillary collection tube connects the retractable needle mechanism to the fluid collection chamber.

5. The medical device of claim 1, wherein the diagnostic chip comprises a micro-assay configured to change electrical impedance when at least one of a chemical, an antigen, an antibody, or a protein is detected in the fluid sample.

6. The medical device of claim 1, wherein the housing further comprises a sensor configured to receive a reading from the diagnostic chip of the diagnostic cartridge.

7. The medical device of claim 6, wherein the housing further comprises an indicator configured to provide an indication based on the received reading from the diagnostic chip.

8. The medical device of claim 6, wherein the housing further comprises a communications module configured to transmit information to a computing device based on the received reading from the diagnostic chip.

9. The medical device of claim 1, wherein at least one of the diagnostic cartridge and the housing further comprises a heating element configured to apply thermal energy to one or more of the retractable needle mechanism, the fluid collection chamber and the diagnostic chip to denature biological contaminants in the fluid sample.

10. The medical device of claim 1, wherein the diagnostic cartridge further comprises a breakable housing containing a caustic solution, the breakable housing configured to rupture after analysis of the fluid sample and wherein the caustic solution is configured to denature any biological contaminants disposed within the retractable needle mechanism, the fluid collection chamber and the diagnostic chip from the fluid sample.

11. The medical device of claim 1, wherein the retractable needle mechanism comprises:
a needle;
a needle housing surrounding the needle;
an injector housing disposed adjacent to the needle housing;
a biasing member disposed within the injector housing, the biasing member configured to apply a biasing force to the injector housing to direct the needle toward a target site;
a retaining housing disposed adjacent the needle housing and opposite the injector housing, the retaining housing configured to engage the needle housing and retain the needle in a retracted position;
a triggering mechanism provided on an exterior of the injector housing,
wherein the injector housing is configured to move toward the needle housing in response to an external force being applied to the triggering mechanism;
wherein the biasing member is configured to provide an increased biasing force to the needle housing in response to the injector housing moving toward the needle housing;
wherein the needle housing is configured to move relative to the retaining housing in response to the increased biasing force exceeding a threshold; and
wherein the needle is configured to pierce the target site in response to the needle housing moving relative to the retaining housing.

12. The medical device of claim 11, wherein the retractable needle mechanism further comprising a retraction groove disposed adjacent the injector housing, the retraction groove being configured to receive and retain the needle in an retracted position in response to the needle piercing the target site.

13. A medical device comprising:
a housing;
a first electrode disposed on the housing, the first electrode configured to contact a first portion of a user;
a second electrode disposed on the housing a distance from the first electrode, the second electrode configured to contact a second portion of the user;
a retractable needle mechanism disposed between the first electrode and the second electrode, the retractable needle mechanism configured to extract a fluid sample; and
a power source configured to provide a voltage between the first electrode and the second electrode to produce electronic anesthesia.

14. The medical device of claim 13, wherein the housing has at least a partial ring structure configured to fit around a body part.

15. The medical device of claim 13, wherein the medical device further comprises a diagnostic chip configured to analyze the fluid sample extracted by the retractable needle mechanism.

16. The medical device of claim 15, wherein the diagnostic chip comprises a micro-assay configured to change electrical impedance when at least one of a chemical, an antigen, an antibody, or a protein is detected in the fluid sample.

17. The medical device of claim 15, wherein the medical device further comprises a sensor configured to receive a reading from the diagnostic chip.

18. The medical device of claim 17, wherein the medical device further comprises an indicator configured to provide an indication based on the received reading from the diagnostic chip.

19. The medical device of claim 17, wherein the medical device further comprises a communications module configured to transmit information to a computing device based on the received reading from the diagnostic chip.

20. The medical device of claim 15, further comprising a heating element configured to apply thermal energy to one or more of the retractable needle mechanism and the diagnostic chip to denature any biological contaminants in the fluid sample.

21. The medical device of claim 15, further comprising a breakable housing containing a caustic solution, the breakable housing configured to rupture after analysis of the fluid sample and wherein the caustic solution is configured to denature any biological contaminants disposed within the retractable needle mechanism and the diagnostic chip from the fluid sample.

22. The medical device of claim 15, wherein one or more of the retractable needle mechanism and the diagnostic chip is disposed within a disposable cartridge removably coupled to the housing.

23. The medical device of claim 13, wherein the retractable needle mechanism comprises:
a needle;
a needle housing surrounding the needle;
an injector housing disposed adjacent to the needle housing;
a biasing member disposed within the injector housing, the biasing member configured to apply a biasing force to the injector housing to direct the needle toward a target site;
a retaining housing disposed adjacent the needle housing and opposite the injector housing, the retaining housing configured to engage the needle housing and retain the needle in a retracted position;
a triggering mechanism provided on an exterior of the injector housing,
wherein the injector housing is configured to move toward the needle housing in response to an external force being applied to the triggering mechanism;
wherein the biasing member is configured to provide an increased biasing force to the needle housing in response to the injector housing moving toward the needle housing;
wherein the needle housing is configured to move relative to the retaining housing in response to the increased biasing force exceeding a threshold; and
wherein the needle is configured to pierce the target site in response to the needle housing moving relative to the retaining housing.

24. The medical device of claim 23, wherein the retractable needle mechanism further comprises a retraction groove disposed adjacent the injector housing, the retraction groove being configured to receive and retain the needle in a retracted position in response to the needle piercing the target site.

25. A medical device comprising:
a retractable needle mechanism configured to extract a fluid sample, the retractable needle mechanism comprising:
a needle;
a needle housing surrounding the needle;
an injector housing disposed adjacent to the needle housing;
a biasing member disposed within the injector housing, the biasing member configured to apply a biasing force to the injector housing to direct the needle toward a target site;
a retaining housing disposed adjacent the needle housing and opposite the injector housing, the retaining housing configured to engage the needle housing and retain the needle in a retracted position;
a triggering mechanism provided on an exterior of the injector housing,
wherein the injector housing is configured to move toward the needle housing in response to an external force being applied to the triggering mechanism;
wherein the biasing member is configured to provide an increased biasing force to the needle housing in response to the injector housing moving toward the needle housing;
wherein the needle housing is configured to move relative to the retaining housing in response to the increased biasing force exceeding a threshold; and
wherein the needle is configured to pierce the target site in response to the needle housing moving relative to the retaining housing.

26. The medical device of claim 25, wherein the retractable needle mechanism further comprising a retraction groove disposed adjacent the injector housing, the retraction groove being configured to receive and retain the needle in a retracted position in response to the needle piercing the target site.

27. The medical device of claim 25, further comprising:
a housing configured to house the retractable needle mechanism, the housing comprising:
a first electrode disposed on the housing, the first electrode configured to contact a first portion of a user;
a second electrode disposed on the housing a distance from the first electrode, the second electrode configured to contact a second portion of a user, and
a power source configured to provide a voltage between the first electrode and the second electrode to produce electronic anesthesia,
wherein the housing houses the retractable needle mechanism between the first electrode and the second electrode.

28. The medical device of claim 27, wherein the housing has at least a partial ring structure configured to fit around a body part of the user.

29. The medical device of claim 27, wherein the medical device further comprises a diagnostic chip configured to analyze the fluid sample extracted from the target site by the retractable needle mechanism.

30. The medical device of claim 29, wherein the diagnostic chip comprises a micro-assay configured to change electrical impedance when at least one of a chemical, an antigen, an antibody, or a protein is detected in the fluid sample.

31. The medical device of claim 29, wherein the medical device further comprises a sensor configured to receive a reading from the diagnostic chip.

32. The medical device of claim 31, wherein the medical device further comprises an indicator configured to provide an indication based on the received reading from the diagnostic chip.

33. The medical device of claim 31, wherein the medical device further comprises a communications module configured to transmit information to a computing device based on the received reading from the diagnostic chip.

34. The medical device of claim 29, further comprising a heating element configured to apply thermal energy to one or more of the retractable needle mechanism and the diagnostic chip to denature any biological contaminants in the fluid sample.

35. The medical device of claim 29, further comprising a breakable housing containing a caustic solution, the breakable housing configured to rupture after analysis of the fluid sample and wherein the caustic solution is configured to denature any biological contaminants disposed within the retractable needle mechanism and the diagnostic chip from the fluid sample.

36. The medical device of claim 29, wherein one or more of the retractable needle mechanism and the diagnostic chip is disposed within a disposable cartridge removably coupled to the housing.

37. A computing device communicatively coupled to a medical device, the computing device comprising a processor and a memory storing computer instructions for controlling the computing device to perform:
receiving, from the medical device, data indicative of a detected at least one of a chemical, an antigen, an antibody, or a protein;
identifying, based on the received data, diagnosis information indicative of a potential medical condition; and
providing, based on the identified diagnosis information, follow-up information related to the potential medical condition.

38. The computing device of claim 37, wherein the follow-up information includes one or more of:
treatment information;
symptom information;
complication information; and
contact information for arranging professional treatment.

39. The computing device of claim 37, further comprising:
sharing, based on a received request, one or more of the received data, the diagnosis information and the follow-up information.

40. The computing device of claim 39, wherein the sharing comprises:
sharing at least one of the received data, the diagnosis information, and the follow-up information via email, SMS message, Website posting, and social media posting.

41. The computing device of claim 39, wherein the sharing comprises:
uploading at least one of the received data, the diagnosis information, and the follow-up information to an electronic medical record database.

42. A computer implemented method for diagnosing a condition, the method comprising:
receiving, from a medical device, data indicative of a detected at least one of a chemical, an antigen, an antibody, or a protein;
identifying, by a computing device, diagnosis information indicative of a potential medical condition based on the received data; and
providing, by a computing device, follow-up information related to the potential medical condition based on the identified diagnosis information.

43. The method of claim 42, wherein the follow-up information includes one or more of:
treatment information;
symptom information;
complication information; and
contact information for arranging professional treatment.

44. The method of claim 42, further comprising:
sharing, based on a received request, one or more of the received data, the diagnosis information and the follow-up information.

45. The method of claim 44, wherein the sharing comprises:
sharing at least one of the received data, the diagnosis information, and the follow-up information via email, SMS message, Website posting, and social media posting.

46. The method of claim 44, wherein the sharing comprises:
uploading at least one of the received data, the diagnosis information, and the follow-up information to an electronic medical record database.

47. A non-transitory computer readable medium having stored therein a program for making a computer execute a method for diagnosing a condition, said program including computer executable instructions for performing the method comprising:
receiving data indicative of a detected at least one of a chemical, an antigen, an antibody, or a protein;
identifying diagnosis information indicative of a potential medical condition based on the received data; and
providing follow-up information related to the potential medical condition based on the identified diagnosis information.

48. The non-transitory computer readable medium of claim 47, wherein the follow-up information includes one or more of:
treatment information;
symptom information;
complication information; and
contact information for arranging professional treatment.

49. The non-transitory computer readable medium of claim 47, further comprising:
sharing, based on a received request, one or more of the received data, the diagnosis information and the follow-up information.

50. The non-transitory computer readable medium of claim 49, wherein the sharing comprises:
sharing at least one of the received data, the diagnosis information, and the follow-up information via email, SMS message, Website posting, and social media posting.

51. The non-transitory computer readable medium of claim 49, wherein the sharing comprises:
uploading at least one of the received data, the diagnosis information, and the follow-up information to an electronic medical record database.
